(19) United States
(12) Patent Application Publication
(10) Pub. No.: US 2011/0054937 A1
(43) Pub. Date: Mar. 3, 2011

(54) Computerized system and method for inputting healthcare clinical orders

(76) Inventor: Vicki Jo Dibble, Summerville, SC (US)

(21) Appl. No.: 12/583,956

(22) Filed: Aug. 28, 2009

(51) Int. Cl. G06Q 50/00; G06F 3/048

(52) U.S. Cl. 705/3; 705/2; 715/809; 715/843; 715/811

(57) ABSTRACT

A customizable system and method are provided for enabling the input of healthcare clinical orders, the method including the steps of: (1) Displaying a service line home page; (2) Providing topic-specific orderset forms for each service line listed; (3) Displaying a selected topic-specific orderset form; (4) Providing pre-populatable medical record information fields on the topic-specific orderset forms; (5) Making multiple clinical order selections available; and (6) Providing contemporary access to an ancillary or miscellaneous order form. This simplified abstract is not intended to limit, and should not be interpreted as limiting, the scope of the claims.

### BACKGROUND OF THE INVENTION

1. Technical Field

The present invention relates to a computerized system and method for enabling healthcare-related clinical order input using closed loop order navigation and a web-based catalog of clinical orders/ordersets customizable to the particular hospital healthcare system.

2. Background Information

Physicians and other busy healthcare providers often see many patients at different locations in a single day. They typically have only a limited amount of time to spend with each patient, and they are inundated with paperwork. Currently, many healthcare providers and staff place clinical orders by finding and manually completing pre-printed clinical order sets or hand written individual orders. These clinical orders are often entered at a later time into another healthcare IT (information technology) application or faxed to appropriate departments within the healthcare system. This manual method of inputting clinical orders is inefficient and has built-in delays and opportunities for error at multiple entry points. Errors often occur at the following points, for example, particularly when the forms are hurriedly and partially completed: interpretation of handwriting, misplacement of paper orders, lost faxed order requests, multiple handoffs, and multiple personnel inputting orders.

Existing software often uses traditional hierarchical menu trees with multiple pull down menus, making use of the software tedious and time-consuming for healthcare providers. The healthcare software orderables that currently exist for clinical order entry do not allow healthcare providers to easily input clinical orders grouped together by patient type and diagnosis.

The system and method of the present invention facilitate inputting of clinical orders into a healthcare application by patient type and diagnosis. The order entry system and interactive method of the present invention permits efficient use of the healthcare provider's time, reduces potential errors, and increases overall patient safety.

### BRIEF SUMMARY OF THE INVENTION

The present invention is a computer implemented, customizable system and method for enabling the input of healthcare-related clinical orders. The new, closed loop order navigation method comprises the steps of: (1) Displaying a customizable service line home page with a list of service lines from which a service line may be selected; (2) Providing a plurality of customizable topic-specific orderset forms for each of the service lines; (3) Displaying the selected topic-specific orderset form; (4) Providing at least one pre-determined, pre-populatable medical record information field on each of the topic-specific orderset forms; and (5) Making multiple, customizable, clinical order selections available to the healthcare provider.

A computerized daily rounding method for enabling the input of healthcare-related clinical orders is also provided herein. It includes the steps of: (1) Providing a “select daily rounding form” option at a point of entry to an order entry collection of a closed loop order navigation system; (2) Providing an option to select a frequently used or miscellaneous order form, or alternatively proceed to one of the service line home pages; (3) Displaying the selected service specific home page, or frequently used or miscellaneous order form; and (4) Providing pre-determined medical record information fields on each of the service line home pages that can be pre-populated with information from the patient record.

The computerized system for enabling the input of healthcare-related clinical orders includes the following, each of which is customizable: (a) a service line home page listing available service lines; (b) a number of service specific home pages available on the service line home page; (c) topic-specific clinical ordersets related to the selected service line; (d) ancillary order forms available on the service line home page; and (e) clinical orders available on each of the topic-specific clinical ordersets or the ancillary order forms. Miscellaneous order forms are preferably also included on the service line home page.

### BRIEF DESCRIPTION OF THE SEVERAL VIEWS OF THE DRAWINGS

A more complete understanding of the invention and its advantages will be apparent from the following detailed description taken in conjunction with the accompanying drawings, wherein examples of the invention are shown, and wherein:

FIG. 1 is a flow diagram showing a method for enabling clinical order input according to the present invention;

FIG. 2 is an illustrative example of a service specific orderset selected from a service specific home page according to the present invention;

FIG. 3 is a flow diagram showing a method for completing the entry of clinical orders or complex order sets according to the present invention;

FIG. 4 is an illustrative example of a service line list or an order entry web page from which a form can be selected according to the present invention;

FIG. 5A is an illustrative example of a service line home page according to the present invention, displaying service specific forms for the service of cardiology, as well as links to frequently used and ancillary order forms;

FIG. 5B is an illustrative example of a service line home page according to the present invention, displaying service specific forms for the service of diabetes management, as well as links to frequently used and ancillary order forms;

FIG. 5C is an illustrative example of a service line home page according to the present invention, displaying service specific forms for the service of hematology and oncology, as well as links to frequently used and ancillary order forms;

FIG. 5D is an illustrative example of a service line home page according to the present invention, displaying service specific forms for the service of vascular surgery as well as links to frequently used and ancillary order forms;

FIG. 6 is an illustrative example of an admission orderset for a selected service according to the present invention, displaying multiple orders from which selections can be made;

FIG. 7 is an illustrative example of a navigation menu according to the present invention, which allows a user to proceed to another form upon completing a current form;

FIG. 8 is a flow diagram illustrating a method for facilitating clinical order input on a daily rounding basis according to the present invention;

FIG. 9 is an illustrative example of a daily rounding form according to the present invention, displaying frequently used forms or allowing a user to navigate to service line home pages;

FIG. 10A is an illustrative example of a complex medication form according to the present invention, which permits the selection and submission of enteral nutrition orders;

FIG. 10B is an illustrative example of an enteral nutrition order form according to the present invention, which permits the calculation of dosages and submission to a pharmacy system;

FIG. 11A is an illustrative example of a multi-modality order form for radiology according to the present invention, wherein common information can be answered once for all imaging modalities;

FIG. 11B is an illustrative example of a diagnostic tab selection of a radiology order form according to the present invention, with helpful drop down menus;

FIG. 11C is an illustrative example of a magnetic resonance imaging (MRI) order tab selection of a radiology order form according to the present invention;

FIG. 11D is an illustrative example of a neuro interventional tab selection of a radiology order form according to the present invention, wherein laboratory results can be displayed if available; and

FIG. 12 is an illustrative example of a mechanical ventilator settings form according to the present invention, which includes typical orders associated with mechanical ventilation.

### DETAILED DESCRIPTION OF THE INVENTION

In the following description, like reference characters designate like or corresponding parts throughout the several views. Also, in the following description, it is to be understood that such terms as “top”, “bottom,” “within,” and the like are words of convenience and are not to be construed as limiting terms. Referring in more detail to the drawings, the invention will now be described.

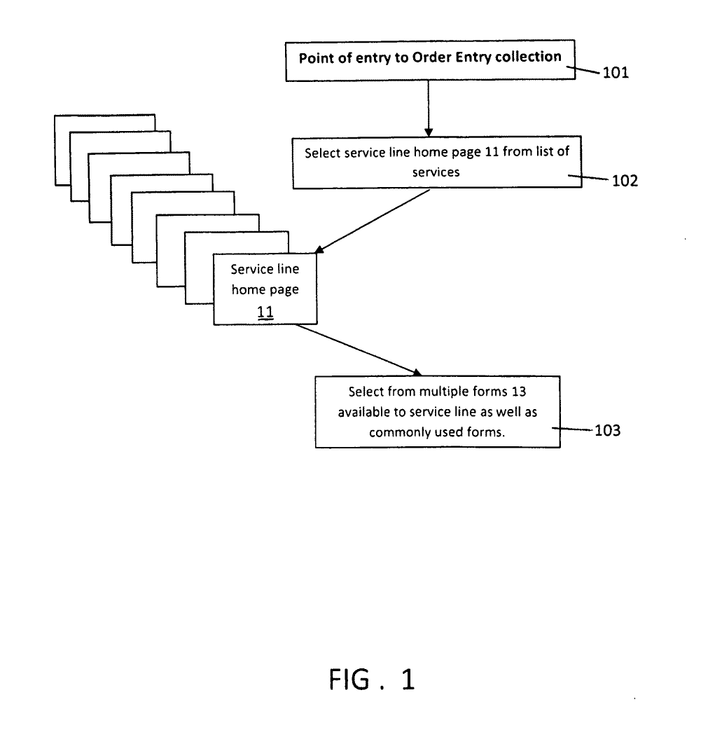

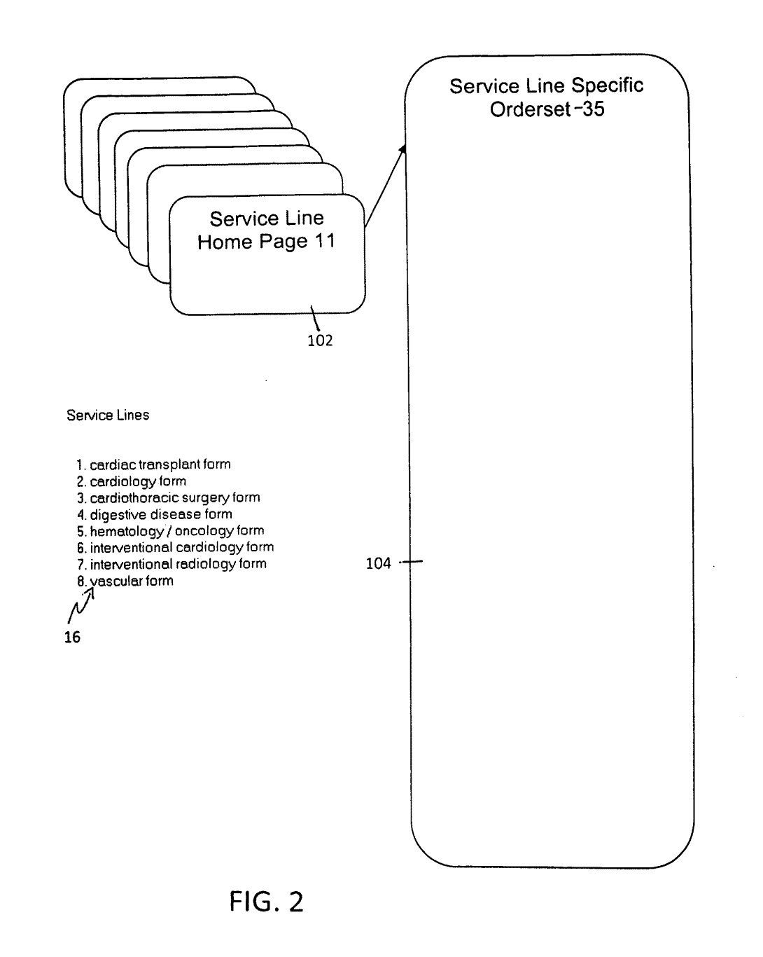

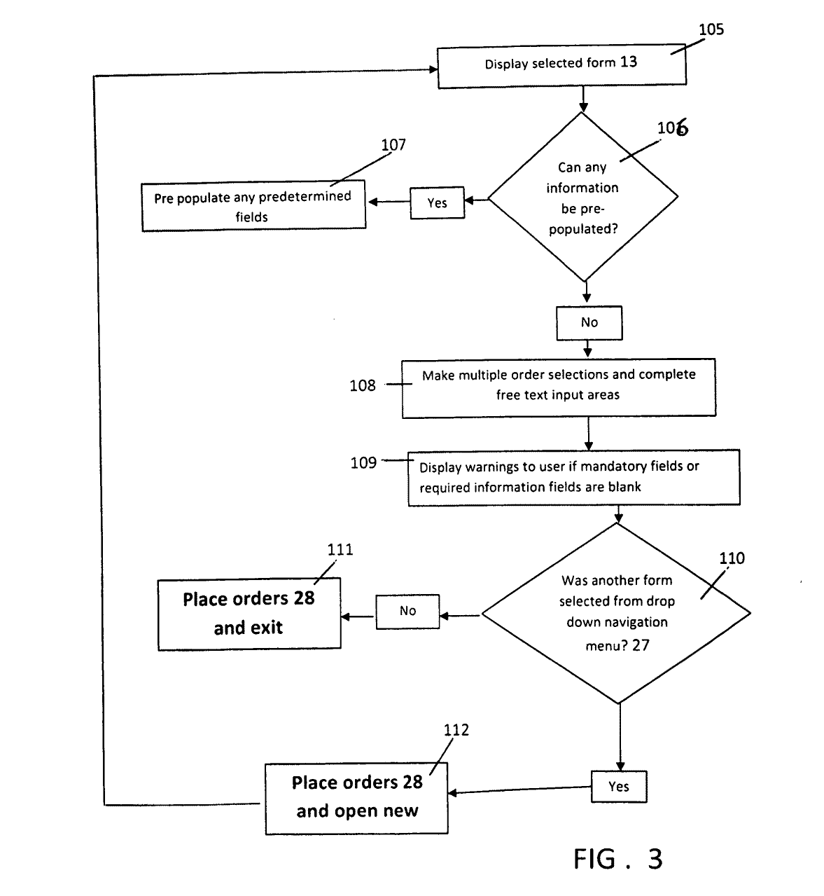

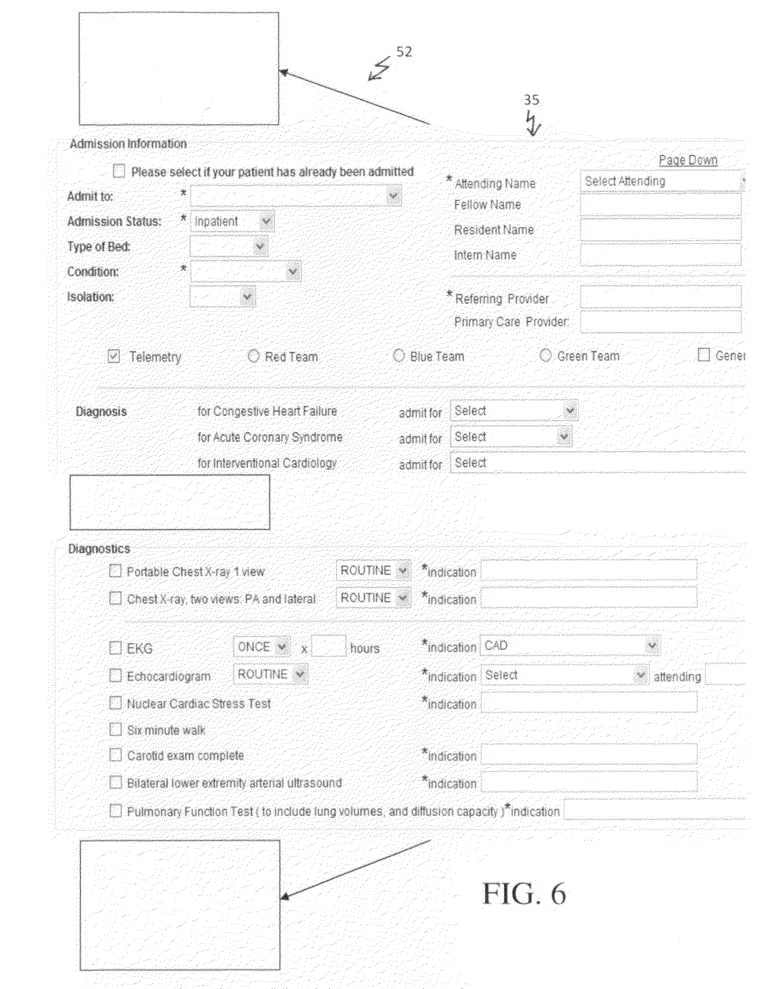

Turning first to FIGS. 1 through 3, a closed loop order navigation method for facilitating the entry of clinical orders, which term is meant to include complete ordersets, includes the following steps after the step of providing a point of entry to the order entry collection, as seen in Block 101 in FIG. 1:
(1) As seen in Block 102 in FIG. 1, displaying a service line home page 11 of the system 10 with a list 16 of service lines for selection (see service line selection area 12; FIGS. 2 and 6).
(2) Providing a plurality of customizable topic-specific orderset forms 35 for each of the service lines.
(3) Displaying the topic-specific orderset form 35 selected by the user;
(4) Providing at least one pre-determined, pre-populatable medical record information field 54 on each of the topic-specific orderset forms 35. The pre-determined medical information field can be pre-populated (hence “pre-populatable”), or filled in, with information stored from the system, such as patient name, age, and birth date, as well as previous lab results, and active orders related to the service specific home pages (see Blocks 106 and 107 in FIG. 3).
(5) As seen in Block 108, making multiple, customizable, clinical order selections available for selection by the healthcare provider (for example, on Admission orderset 52 in FIG. 6). Authorized healthcare facility personnel may customize these order forms by adding and/or subtracting orders, text areas, and/or drop-down menus.

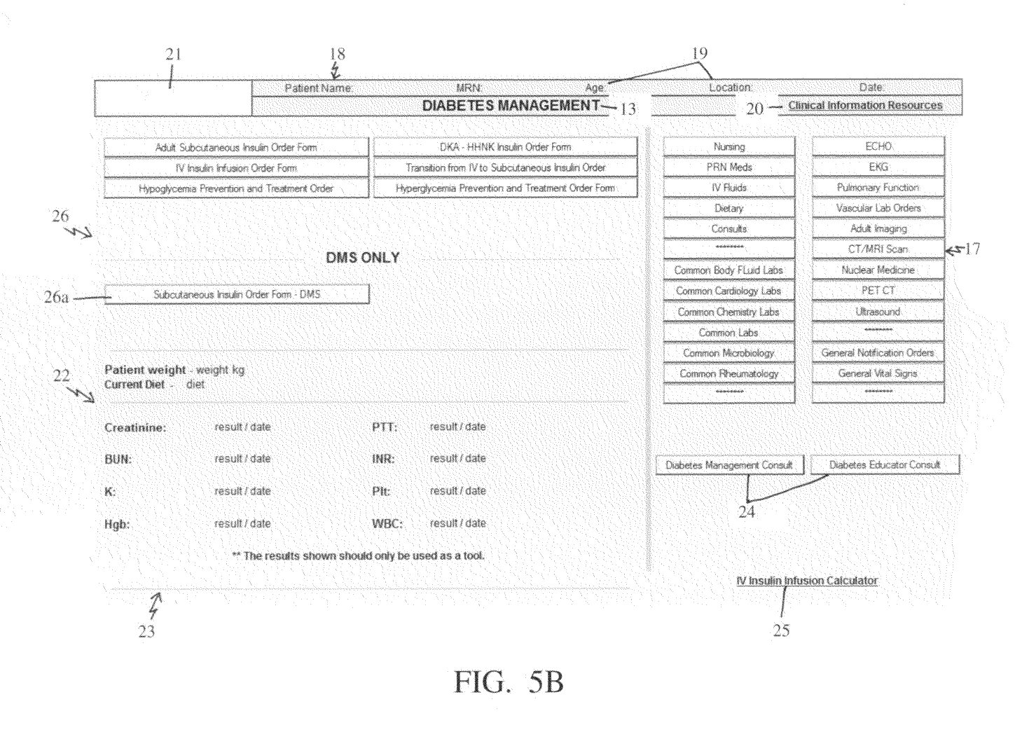

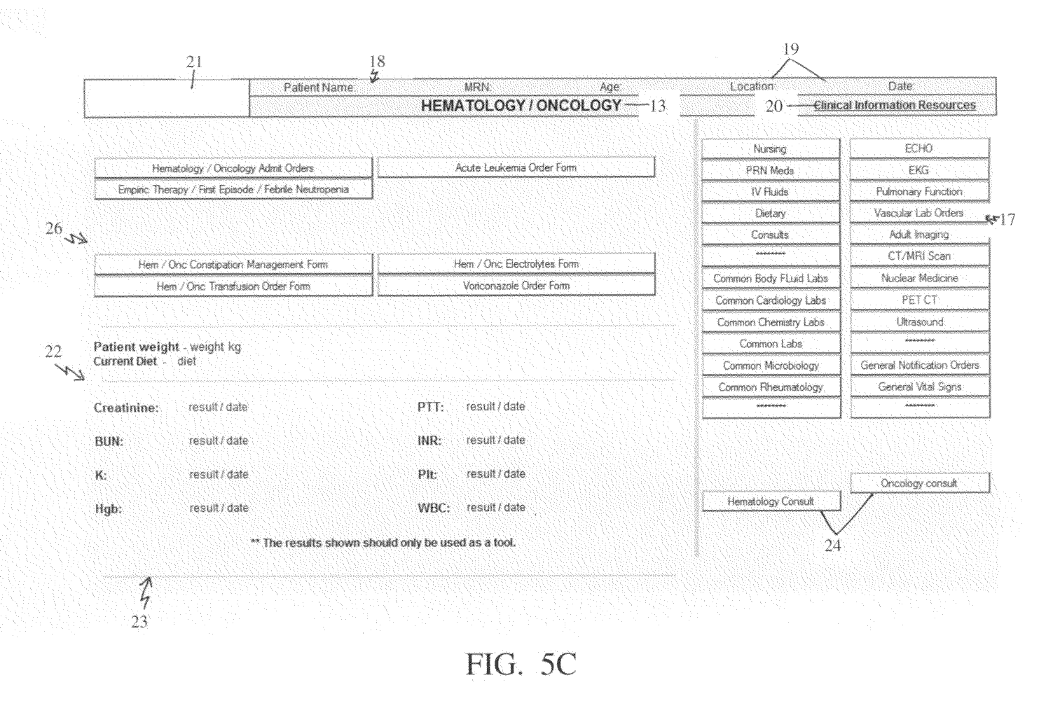

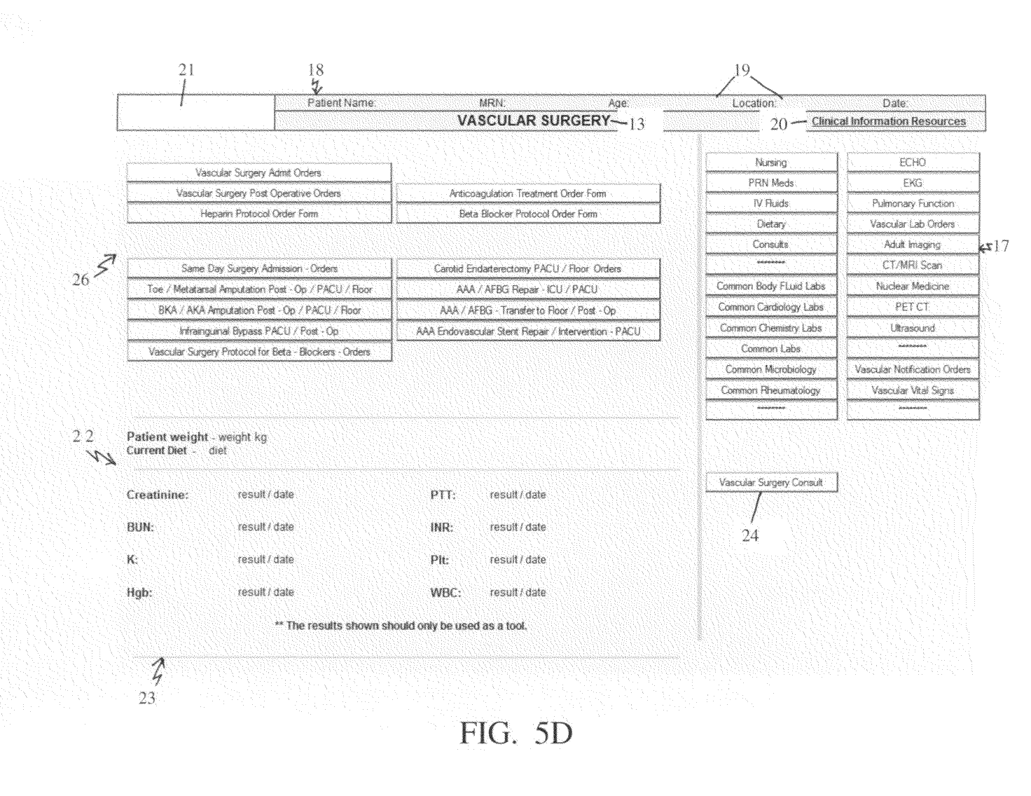

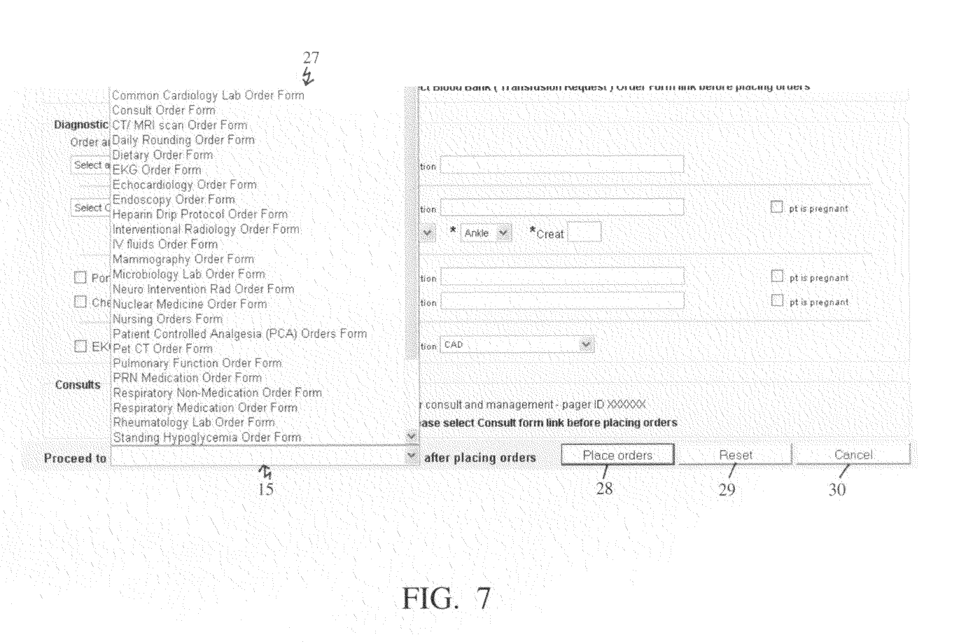

Continuing with FIGS. 1 through 3, the preferred method preferably also includes the steps after step (5) of:
(6) As shown in Block 103 in FIG. 1, providing a number of ancillary order forms 14 available for each of the service lines of the system 10, as well as frequently used or miscellaneous order forms (see frequent/miscellaneous/ancillary form selection area 17 in FIG. 5A) from which the user makes a selection. The customized, miscellaneous order forms may include a consult form (see consult order space 24 in FIG. 5A) or a nursing order form. These order forms are available contemporaneously, meaning here that they can be selected within the same session on the computer (all within the same system 10). The user need not leave the system 10 to access an ancillary or miscellaneous order form, for example. A particular user need not select more than one type of order form, though.
(7) Turning to Block 105 in FIG. 3, displaying the ancillary order form 14 selected by the user.
(8) Making free text areas 53 on the topic specific orderset forms available for adding specific details to a selected clinical order (see Block 108). The free text areas are customizable. For example, detailed wound care for the patient may be inputted in an appropriate free text input area 53. Free text areas 53 need not be filled in, except where a clinical order may require additional details.
(9) Displaying a warning if required information fields are left unanswered (Block 109 in FIG. 3). A hospital or other healthcare facility may require that certain healthcare information be completed at the time of order entry. The warning may be visual, such as a cautionary message on a pop-up window.
(10) Providing an option for selecting an additional, miscellaneous form 17 from a drop-down navigation menu 27, per Blocks 110 and 111 in FIG. 3 (also see FIG. 7).
(11) Providing a Place Orders button 28 (see bottom of FIG. 7), in which clinical orders can be placed, and then permitting the user to exit without selecting an additional form from the drop-down navigation menu 27, per Block 111 of FIG. 3.

As illustrated in FIG. 2, the service line can be selected on the service line home page 11 from among the following examples: cardiac transplant form; cardiology form; cardiothoracic surgery form; digestive disease form; hematology/oncology form; interventional cardiology form; interventional radiology form; and vascular form. Other service lines may be included instead of or in addition to these service lines. The service specific orderset 26 is then entered, as seen in Block 104 in FIG. 2.

The computerized system 10 is an example of a system for enabling the input of clinical orders into a healthcare application according to the present invention. This customizable closed loop order navigation system 10, which is seen in the figures herein, includes the following:
(a) a service line home page 11 listing available service lines;
(b) a number of service specific home pages 13 (e.g., cardiology, diabetes management, etc.) available on the service line home page 11;
(c) topic-specific clinical ordersets 35 related to the selected service line and relevant to the patient's condition and diagnosis;
(d) ancillary order forms 14 (e.g., lab, radiology, EKG, etc.) available on the service line home page 11; and
(e) clinical orders 17 available on each of the topic-specific clinical ordersets 35 or the ancillary order forms 14. All of these home pages, ordersets, forms, and clinical orders can be customized to suit the particular healthcare environment in which they are used. The system 10 enables placement of multiple clinical orders in one uninterrupted session, or without unnecessary/intermediate exiting from the system 10. By “intermediate exiting” is meant that the user need not exit the system 10 until he or she is finished, or chooses to end, the computer session. With the present system 10, it is not necessary for a user to exit the system 10 in order to search for the forms they need.

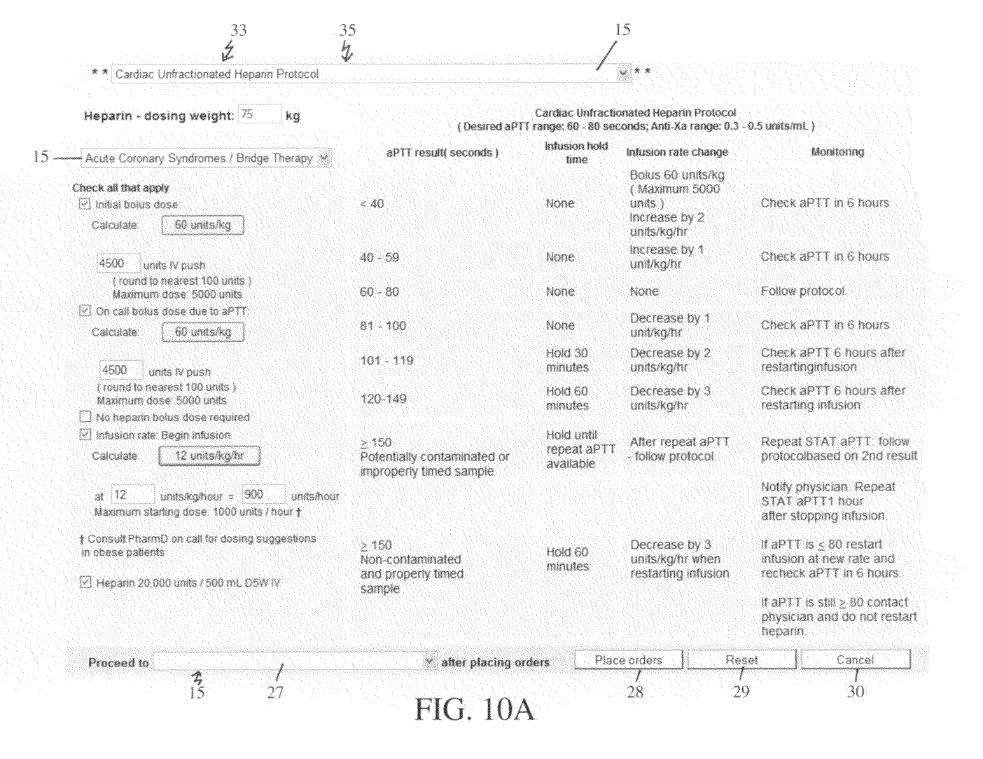

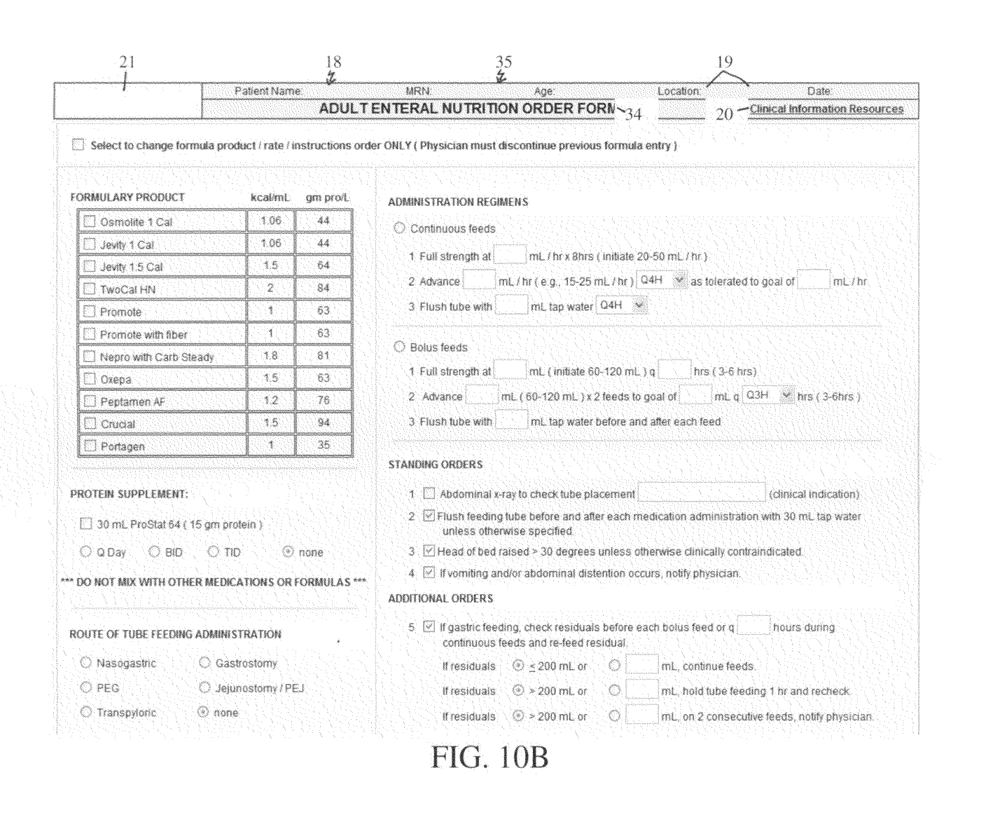

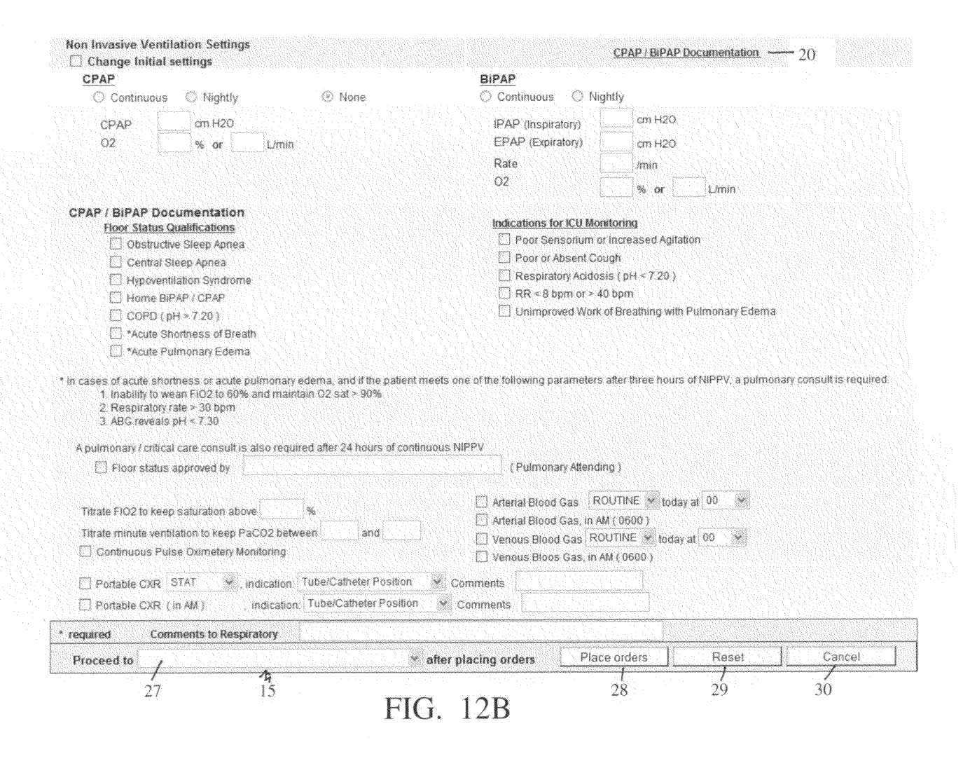

A conventional search engine can be used to locate the present system. This system can be used in collaboration with any suitable existing order entry system. The point of entry (see FIGS. 10 and 12) for the present system 10 may reside in a hospital healthcare information system, such as an external order entry application. One way of accessing the service line home page is to make a service line home page button available on the external order entry system. An alternate way of accessing the service line home page is to perform a search for the service line home page at the point of entry.

The closed loop order navigation system 10 may be implemented through the use of a secure healthcare network. The system 10 can be accessed by a plurality of users on personal computers, cellular telephones, personal digital assistants, or the like, which have access to the secure network. The users are intended to be physicians or other healthcare providers and the plurality of personal computers or the like are generally at hospitals and in other healthcare environments.

A preferred computerized clinical order facilitation system 10 herein facilitates clinical order entry by providing predefined variables, calculations, and drop-down lists. The closed loop order navigation system 10 can also facilitate data entry by permitting input of free form text into at least one information field of a clinical order.

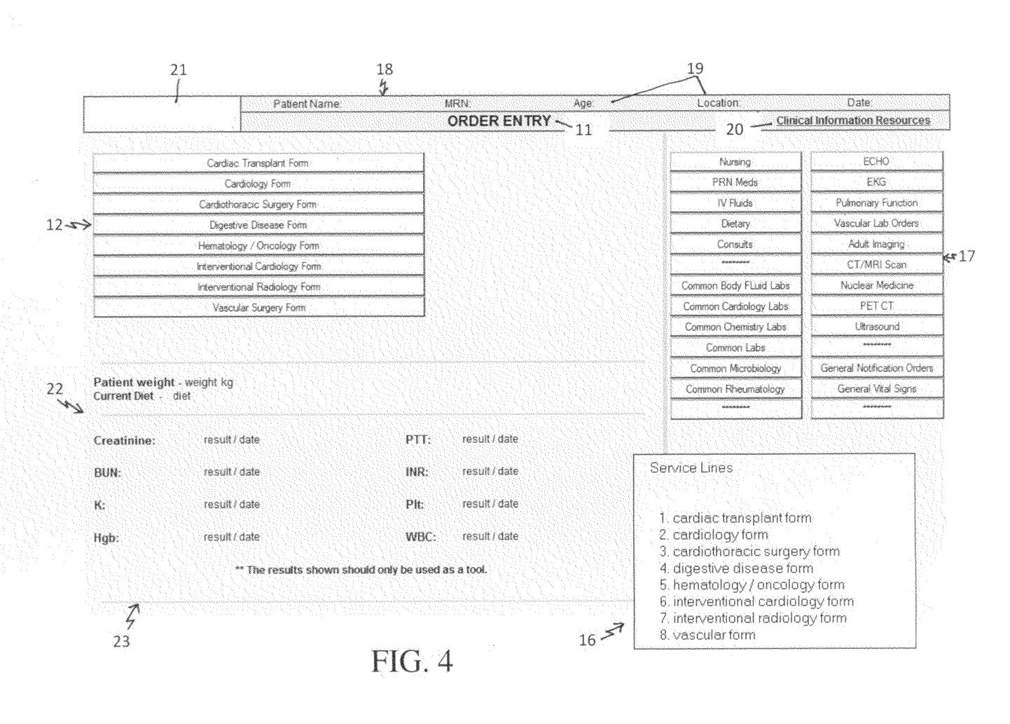

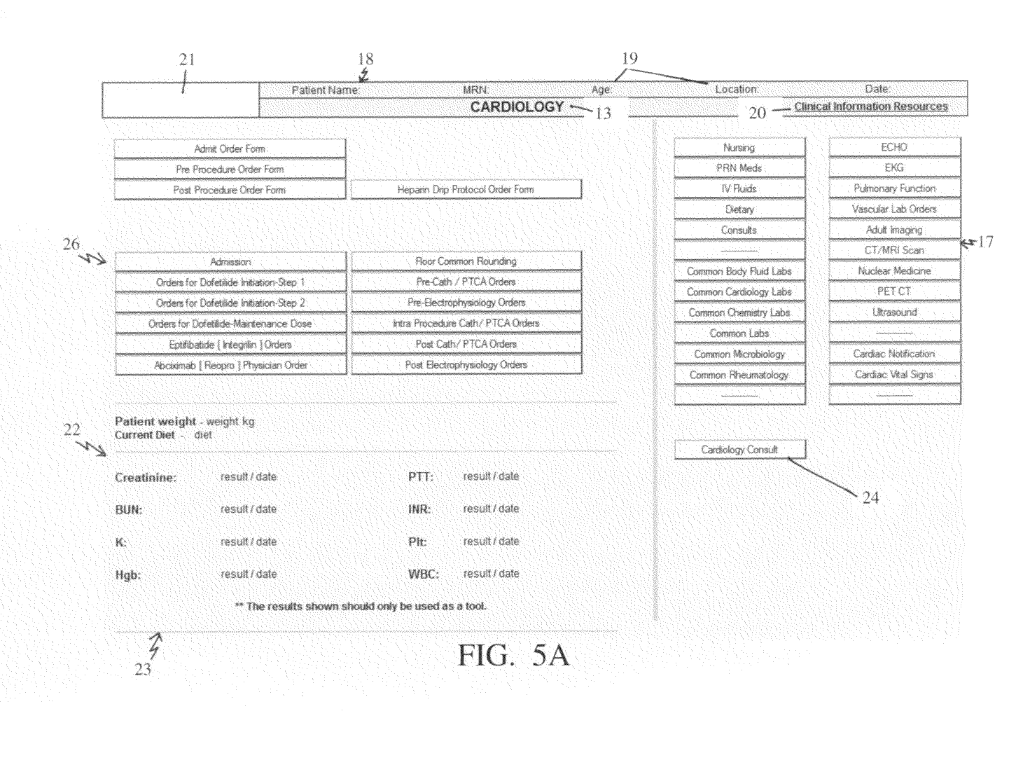

The preferred computerized clinical order facilitation system 10 herein uses stored information to pre-populate information fields. The stored information used to pre-populate the order form can be, but is not limited to, stored clinical information, such as patient allergies, height and weight, vital signs, and laboratory test results (see pre-populatable patient data field 22 and pre-populatable lab results field 23 in FIG. 4).

A preferred computerized clinical order facilitation system 10 herein facilitates order form selection by selecting a service line home page from a list provided or selecting a service line home page from the order entry web page. In this way, the system can provide the user with a list of forms that is related to the particular service line.

A preferred computerized clinical order facilitation system 10 facilitates clinical order entry, offering many clinical order selections within one form. This allows the user to complete a large number of clinical orders sequentially in a timely and efficient way, saving the healthcare provider's time and expediting the patient's care. The system 10 is unique in that the user need not exit the system in order to locate the next order form, then do the same for a third order form. Permitting the user to skip several steps results in significant time savings. For example, one healthcare provider/user may wish to order lab results for one patient, while another user at the same hospital may wish to specify mechanical ventilator settings for a second patient, and not order labs.

A preferred computerized clinical order facilitation system 10 uses a web page format. This makes it easier for the user to visualize all the orders. This computerized clinical order facilitation system 10 provides a drop-down menu 15 of many available, customizable, frequently used order forms, which allows the user to navigate from one form to another without having to exit the system. This enables the healthcare provider to access and place a variety of clinical orders relevant to the patient's condition and diagnosis. The clinical order facilitation system 10 makes the placing of orders more time efficient for the healthcare provider. The healthcare provider is then free to visit the patient, and undertake other tasks. By “frequently used” is meant those order forms that are most often used by healthcare practitioners. Since the system 10 is customizable, what is frequently used can determined by statistics kept by the appropriate healthcare facility department, possibly by a knowledge management team at the healthcare facility/organization.

The closed loop order navigation system 10 does not analyze recorded patient information, nor does it recommend clinical orders, guidelines, interventions, or treatment and diagnosis for a particular patient. The system 10 does not make decisions for the healthcare provider. The system 10 does not analyze and organize patient information for the purpose of making a comprehensive medical history or a patient progress note. The system 10 does not compile, measure, or disseminate data. The system 10 is intended for use by healthcare providers but not by patients themselves. Patients are not intended to input to the system 10. The system 10 is not itself a stand-alone application intended to interface with other applications, although the system 10 can live within such an application. The system 10 is not intended for placing a single type of order, such as radiology or pharmaceuticals, unless ordered from within a topic-specific orderset form. Various types of orders (including medical procedures), such as cardiology or radiology-related orders, can be placed in a single session on the system 10.

Importantly, the closed loop order navigation system 10 allows a healthcare provider to place a multiplicity of various types of clinical orders without having to exit the system 10. The closed loop order navigation system 10 may include the additional steps of navigating to a second, ancillary order form or frequently used or miscellaneous order form 17 within the system 10, selecting and displaying particular order forms, and placing orders (see FIG. 7). Thus, a user can navigate to one order form when selected, place orders, and stop, or proceed to second and third, etc. order forms, placing orders along the way. Also, the user need not take the time to type in each individual order; the user may instead simply click on the appropriate buttons. One or many orders may be selected.

The preset, computer implemented ordering system 10 offers a multiplicity of clinical orders from which to choose. Order selections relate to various hospital service lines, such as cardiology, nephrology, and hematology, and ancillary departments, such as lab, radiology, and EKG, and various medical specialties. All clinical orders for a particular patient can be placed using a single form, without having to exit the system.

The medical provider may select clinical orders suitable for a particular patient from a single form, such as medications, activities, lab procedures, diagnostic testing, and nursing care. This method reduces the time that is necessary for the clinician to input multiple clinical orders. Hospital personnel receive orders in a timely and clear manner, resulting in improved patient care. Reduction of order-related errors results in improved patient safety. Orders entered can be retrieved or utilized for data collection. Overall, the system 10 results in improved patient, patient family, and healthcare provider satisfaction.

The computerized ordering system 10 includes external links to medical research/pharmaceutical information on medications and medical treatments, in case the healthcare professional is seeking additional information about a particular medication or treatment, or alternatives for it. Links to the system 10 are customizable, according to the user's preference.

FIGS. 4-7 provide examples of forms according to the present computerized clinical order facilitation method. An Order Entry web page form from which the user can make a form selection is shown in FIG. 4. A service line list 16 as shown on FIG. 4 (lower right) can alternatively be used to make a form selection.

A frequently used/miscellaneous/ancillary form selection area 17 is also shown on FIG. 4 (upper right). The user uses this form selection area 17 to select individual forms from which they can make multiple clinical order selections related to the topic or topics that are selected (e.g., nursing, ECHO, PRN meds, EKG).

The Order Entry form, which is a service specific home page 13, also includes certain medical record information fields 54 that can be pre-populated with information from the patient's medical records, such as a pre-populatable patient information field 18 (see top of FIG. 4). The patient information field 18 multiple information spaces 19 for entry of patient information, such as patient name, age, birth date, gender, location in the healthcare facility, current date, etc. The patient information field 18 preferably also includes space for the logos 21 of the particular healthcare facility or organization using the system 10. The pre-populatable patient information field 18 also preferably includes a Clinical Information Resources link 20 for linking to an external resource or information page (e.g., external links for researching evidence based medicine). The Clinical Information Resources link 20 can be customized by the user.

The Order Entry form 13 preferably also includes a second medical record information field 54: a pre-populatable, patient data field 22 for viewing data charted on a particular patient, such as patient weight and current diet (see FIG. 4). This patient data field 22 is pre-populated by the system from the patient's electronic record.

A third medical record information field 54 in the Order Entry form 13 is preferably a pre-populatable lab result field 23 for visually displaying previous lab results for the particular patient (see FIG. 4). This lab result field 23 is pre-populated by the system from the patient's electronic record.

A service line home page 11, which displays service specific forms and links to frequently used forms for the service of cardiology, is shown in FIG. 5A. The service line home page 11 includes the pre-populatable patient information field 18. The Cardiology service line form 12 preferably also includes the second pre-populatable, patient data field 22 for viewing data charted on a particular patient, and the pre-populatable lab result field 23 for visually displaying previous lab results for the particular patient. The Cardiology service line form 12 also includes a service specific orderset area 26 that lists ordersets specific to the service line of Cardiology, such as Admit Order Form, Pre Procedure Order Form, Post Procedure Order Form, Heparin Drip Protocol Order Form, and Admission Outline. Also included is a one-click Consult Order space 24 for ordering a healthcare consult for the particular service line (Cardiology in FIG. 5A). The system 10 may include one-click frequently used clinical orders, such as laboratory tests or nursing orders. By “one-click” is meant that the user simply clicks the appropriate button, which quickly places the subject single order.

A service line home page 11 displaying service specific forms as well as links to frequently used forms for the service of diabetes management is shown in FIG. 5B. Along with the features that are also displayed on FIG. 5A, the Diabetes Management Service (DMS) order form, which is a service specific home page 13, includes a one click External Calculator Link space 25 for accessing a calculator for calculating medication dosages. The service specific orderset area 26 includes a special orderset space 26a shown in FIG. 5B (e.g., subcutaneous insulin order form-DMS) that should only be utilized by the DMS service line specialist, such as an endocrinologist.

A service line home page 11 displaying service specific forms and links to frequently used forms for the service of hematology/oncology is shown in FIG. 5C. It includes features in common with FIGS. 5A and 5B, a pre-populatable patient information field 18, a service specific orderset area 26, a frequently used/ancillary/miscellaneous form selection area 17, a pre-populatable patient date field 22, a pre-populatable lab result field 23, and a Consult Order space 24.

A service line home page 11 displaying service specific forms and links to frequently used forms for the service of vascular surgery is shown in FIG. 5D. It includes features in common with FIG. 5A-C, a pre-populatable patient information field 18, a service specific orderset area 26, a frequently used/miscellaneous/ancillary form selection area 17, a pre-populatable patient date field 22, a pre-populatable lab result field 23, and a Consult Order space 24

An Admission orderset 52 for a selected service line, such as Cardiology, displays multiple clinical orders from which the healthcare provider may make one or more order selections, as shown in FIG. 6 (also see FIG. 2). Clinical order selections are made using the drop-down menus, free text areas, check boxes, and radio buttons, as shown in the expanded boxes of FIG. 6.

Referring to the lower portion of the form shown in FIG. 7, a drop down navigation menu 27 allows a user to select another form to which the user will be directed upon placing the clinical order. The drop down navigation menu 27 may include frequent lab forms, radiology forms, respiratory forms, etc. An Admission orderset form 52, as well as other forms (except for the service specific home pages), includes a set of three buttons. First, a Place Order button 28 is included for placing the user's order selections. Second, a Reset button 29 is included for clearing the information on the page and allowing the user to start over (e.g., where the user has made errors). Lastly, the Cancel button 30 is available for canceling the order session for the selected patient. The Cancel button 30 may be used, for example, when the wrong patient has inadvertently been selected by the user at the start of the session, or where an emergency arises and the user must leave the order session.

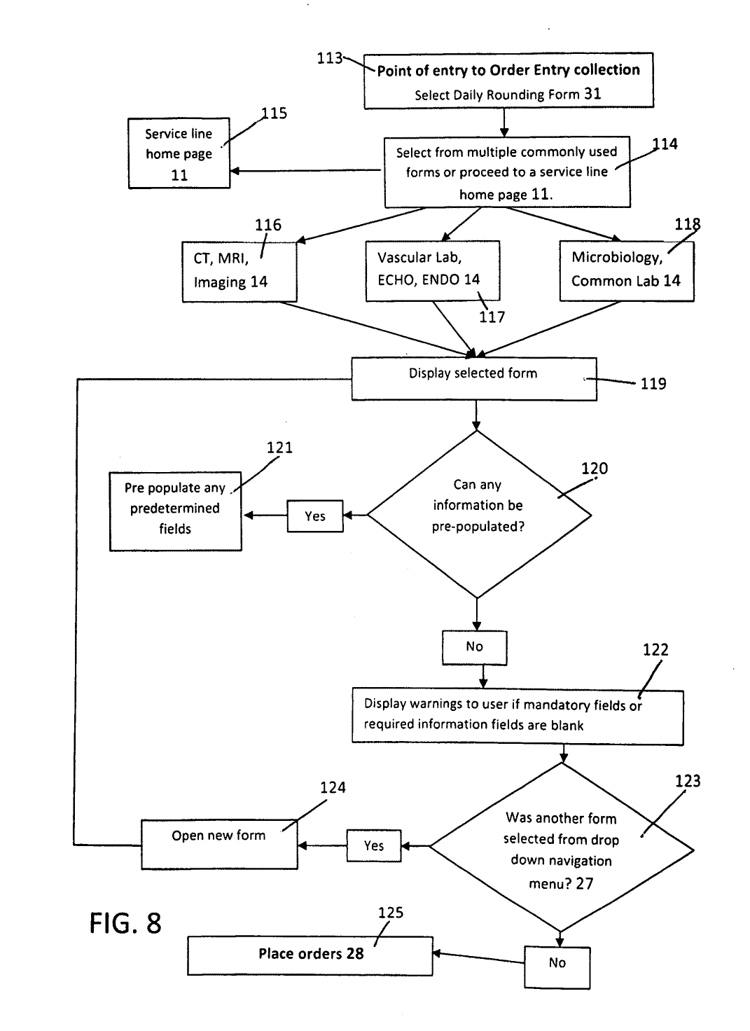

A method herein for facilitating clinical order input on a daily rounding basis includes the following steps:
(1) As seen in Block 113 in FIG. 8, providing a “daily rounding” form option 31 at a point of entry to the order entry collection of the closed loop order navigation system 10. Alternatively, the daily rounding form 31 can be on the service line home page 11.
(2) As seen in Block 114, providing an option to select among multiple order forms (see frequent/ancillary/miscellaneous form selection area 17), or alternatively proceeding to the service line home page 11 where a service line (see service line selection area 12) may be selected (Block 115), and then displaying the service specific home page 13 (if that option has been selected), per Block 119 in FIG. 8.
(3) Providing at least one pre-determined patient information space 19, such as patient name, age, birth date, etc., that can be pre-populated with information from the stored patient electronic record, such as previous lab results, active patient orders, per Blocks 120 and 121.
   This computerized clinical order facilitation method preferably also includes the steps of:
(4) Displaying a warning if mandatory information fields are left unanswered (Block 122). A hospital may require that certain questions be asked of a patient on admission. For example, responses to questions related to clinical history may be required. The warning may be visual, such as a cautionary message on a pop-up window.
(5) Providing a number of customizable, frequently used and/or ancillary order forms 14, per Block 123, or miscellaneous order forms, such as a consult order form, a nursing order form.
(6) Providing a Place Orders button 28, in which clinical orders can be placed and permitting the user to exit without selecting additional forms from the drop-down menu 15, per Block 125 in FIG. 8.
(7) If an additional clinical order form is selected from the drop-down navigation menu 27, the new clinical order form will display, per Block 124, after selecting the Place Orders button 28.

The selectable frequently used/miscellaneous form options may include the following, for example: (a) Computerized Tomography (CT), Magnetic Resonance Imaging (MRI), Imaging, per Block 116, (b) Vascular Lab, echocardiogram (ECHO), ENDO, per Block 117, and/or (c) Microbiology, Common Lab, per Block 118.

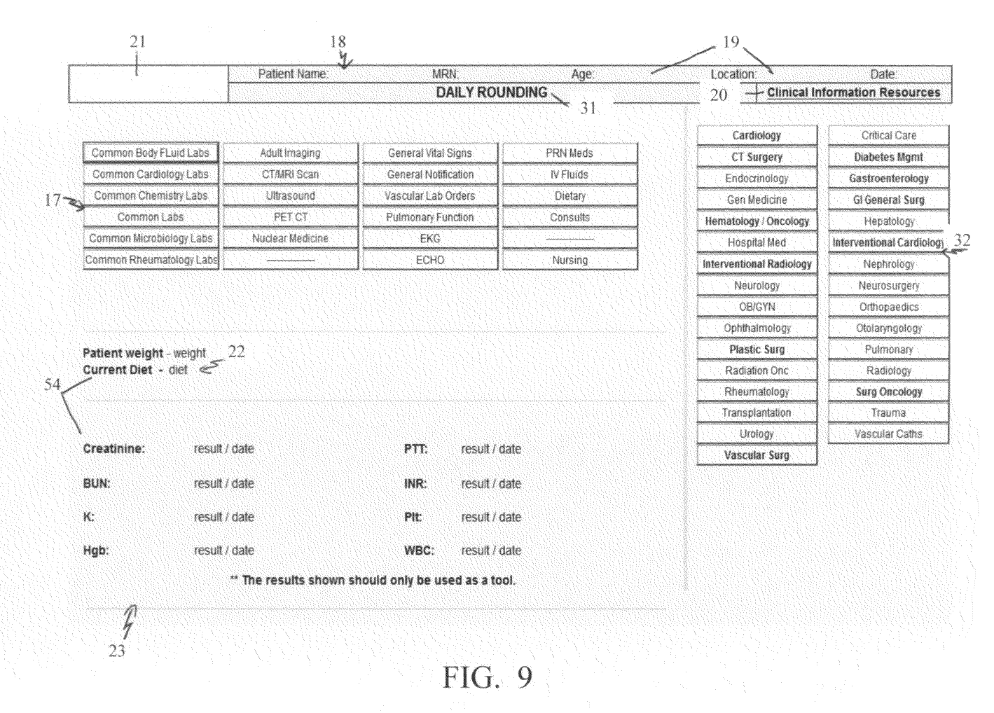

FIGS. 9-12 provide examples of printouts according to the present computerized clinical order facilitation method. Referring to FIG. 9, a daily rounding form 31 displays frequently used/miscellaneous forms 17 and/or allows a user to navigate to the service line home page 11. As described herein, the daily rounding form 31 includes a pre-populatable patient information field 18, a frequent/miscellaneous/ancillary form selection area 17, a pre-populatable patient data field 22, and a pre-populatable lab result field 23. The patient information field 18 includes the organization's logos 21, a patient information space 19, and an external Clinical Information Resources link 20. The daily rounding form 31 also includes a service line list area 32 for navigating to the service line home page 11.

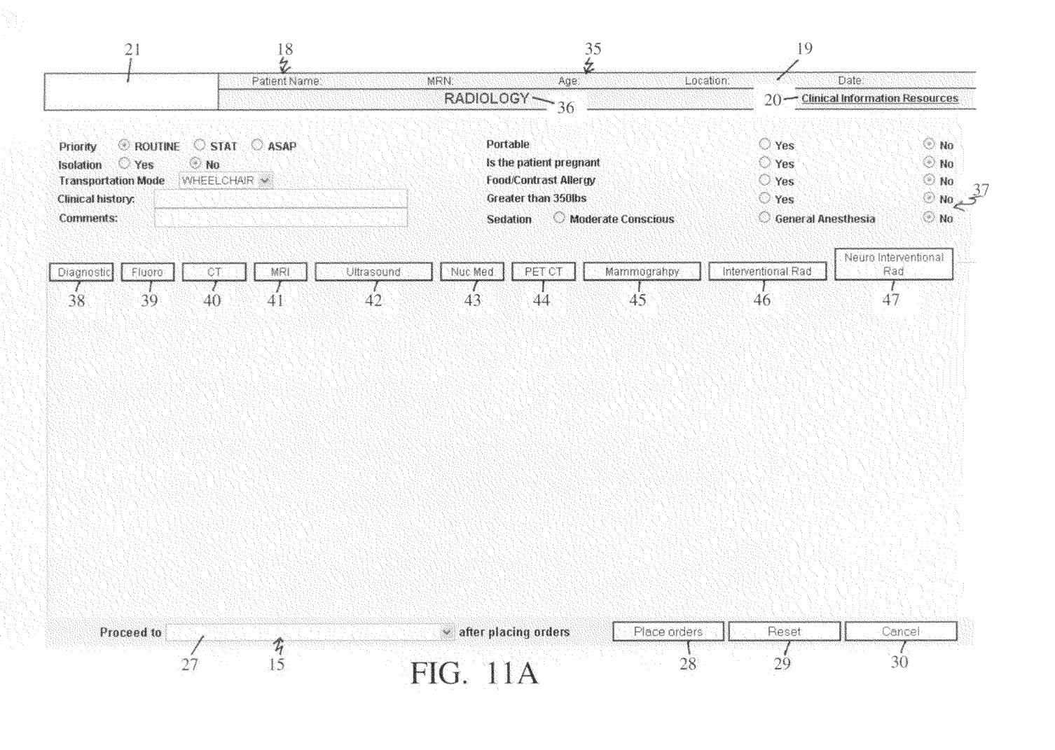

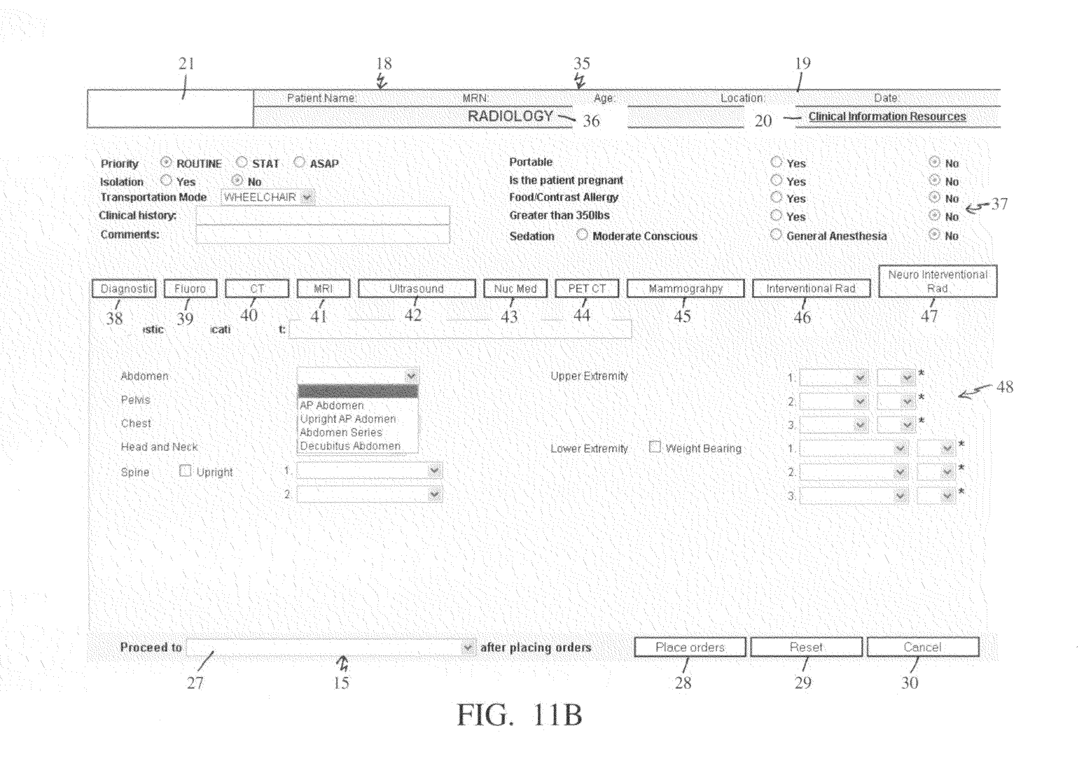

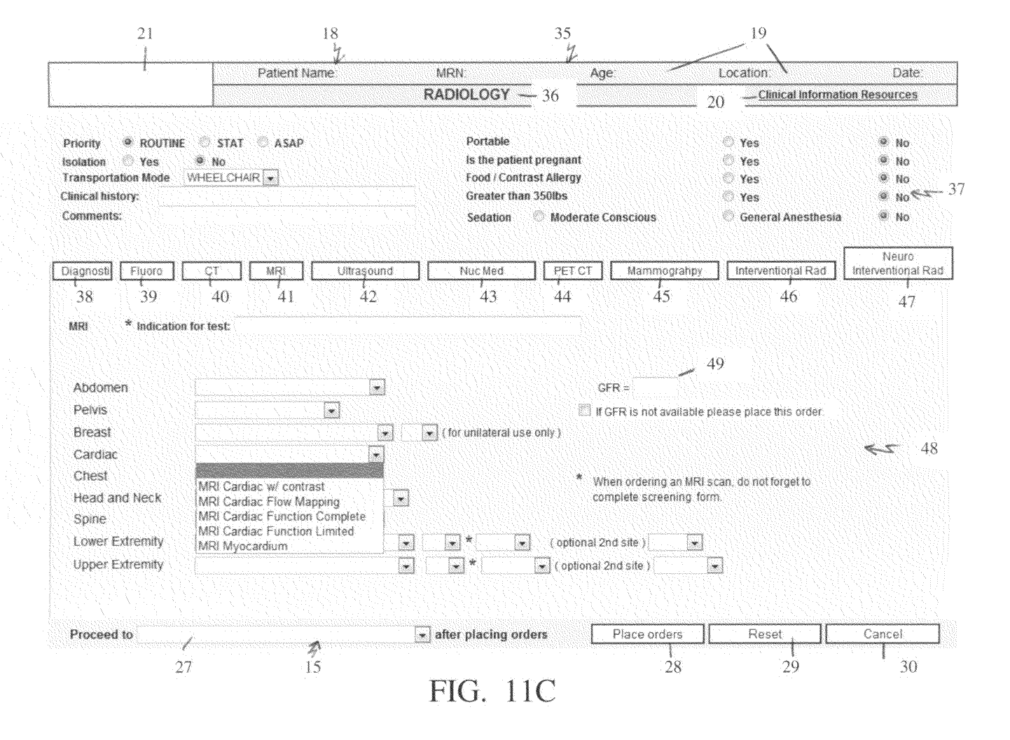

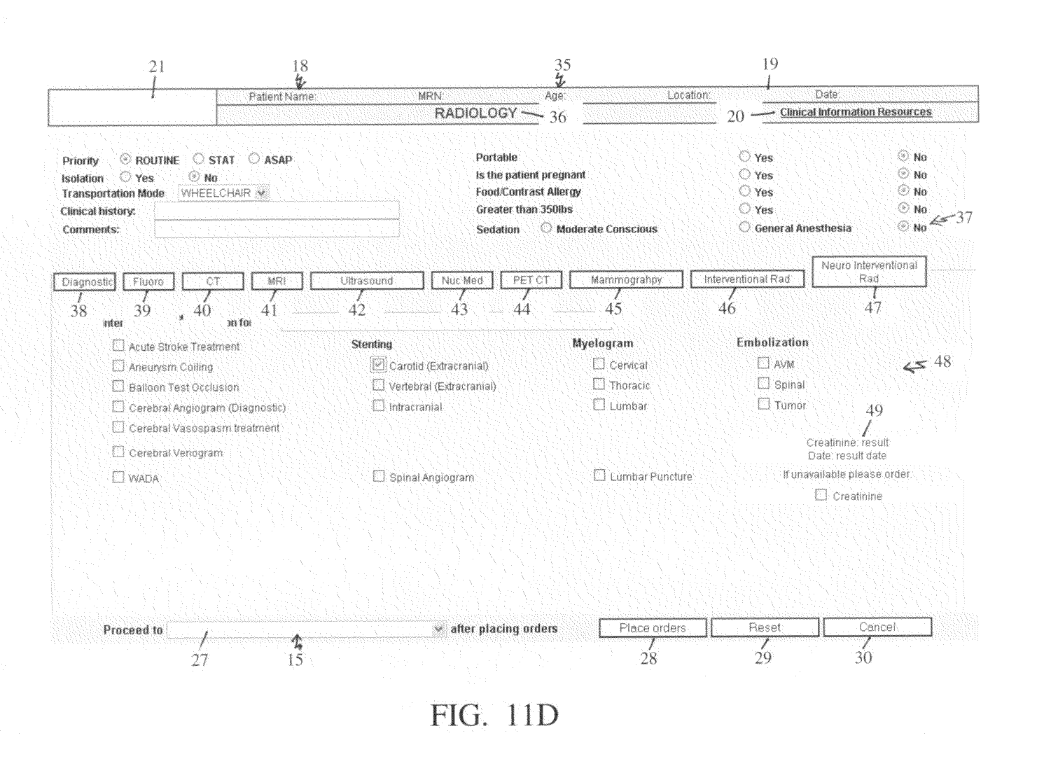

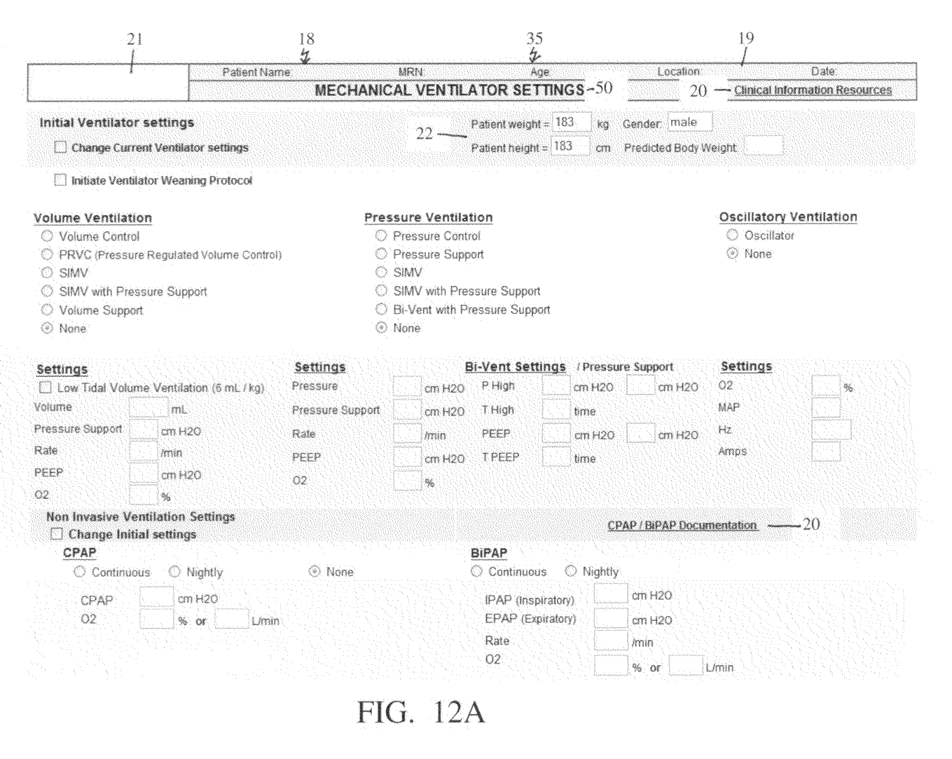

The system 10 also includes topic-specific ordersets 35, such as the complex medication form 33 and the enteral nutrition order form 34 shown in FIGS. 10A and 10B, respectively, the multi-modality radiology order form 36 shown in FIG. 11A, and the mechanical ventilator settings form 50 shown in FIG. 12. First, the complex medication form 33 permits a user to calculate dosages and submit clinical orders to a pharmacy system (see FIG. 10A). Secondly, the enteral nutrition order form 34 shown in FIG. 10B permits the healthcare provider/user to place nutrition orders. They can be customized to suit the needs of the particular healthcare organization.

When the multi-modality radiology order form 36 is selected, common information can be answered once for all imaging modalities in a common required information area 37, as seen in FIG. 11A. Various imaging modalities may be selected using the following buttons: diagnostic tab selection 38, fluoroscopy (“Fluoro”) tab selection 39, Computerized Tomography (CT) tab selection 40, MRI (Magnetic Resonance Imaging) tab selection 41, ultrasound tab selection 42, nuclear medicine (“Nuc Med”) tab selection 43, PET CT (Positive Electron Tomography Computerized Tomography) tab selection 44, mammography tab selection 45, interventional radiology tab selection 46, and neuro (Neurology) interventional radiology tab selection 47. The diagnostic tab selection 38 of a radiology order form 36 (see middle of form) is depicted in FIG. 11B. MRI tab selection 41 of a radiology order form 36 is shown in FIG. 11C. FIG. 11D shows an example of a neuro interventional tab selection 47 of the radiology order form 36.

When a modality selection tab is selected, the modality specific area 48 will display clinical orders for the selected modality. Where laboratory results are pertinent and required for imaging, a laboratory results area 49 is provided within the modality specific area 48, as seen in FIG. 11D. If such lab results are available, they can be pre-populated from the system.

Lastly, a mechanical ventilator settings form 50 is shown in FIG. 12. The mechanical ventilator settings form 50 includes orders typically associated with mechanical ventilation for healthcare purposes. The mechanical ventilator settings form 50 includes a customizable Clinical Information Resources link 20. It preferably also includes a pre-populatable patient data field 22 for viewing data charted for a particular patient, such as patient weight, height, and gender. This patient data field 22 is pre-populated by the system 10 from the patient's electronic record. Another type of miscellaneous order form is a Do Not Resuscitate (DNR).

From the foregoing it can be realized that the present invention may be easily and conveniently utilized as a computer implemented system and method for enabling the input of healthcare-related clinical orders. It is to be understood that any dimensions given herein are illustrative, and are not meant to be limiting.

While preferred embodiments of the invention have been described using specific terms, this description is for illustrative purposes only. It will be apparent to those of ordinary skill in the art that various modifications, substitutions, omissions, and changes may be made without departing from the spirit or scope of the invention, and that such are intended to be within the scope of the present invention as defined by the following claims. It is intended that the doctrine of equivalents be relied upon to determine the fair scope of these claims in connection with any other person's product which fall outside the literal wording of these claims, but which in reality do not materially depart from this invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

### BRIEF LIST OF REFERENCE NUMBERS USED IN THE DRAWINGS

10 clinical order input system
11 service line home page
12 service line selection area
13 service specific home pages
14 ancillary order forms
15 drop-down menu
16 service line list
17 frequent/miscellaneous/ancillary form selection area
18 pre-populatable patient information field
19 patient information spaces
20 Clinical Information Resources link
21 logos
22 pre-populatable patient data field
23 pre-populatable lab result field
24 Consult Order space
25 external calculator link space
26 service specific orderset/area
27 drop down navigation menu
28 Place Orders button
29 Reset button
30 Cancel button
31 daily rounding form
32 service line list area
33 complex medication form
34 enteral nutrition order form
35 topic-specific orderset
36 radiology order form
37 common required information area
38 diagnostic tab selection
39 fluoro tab selection
40 CT tab selection
41 MRI tab selection
42 ultrasound tab selection
43 nuclear medicine tab selection
44 PET CT tab selection
45 mammography tab selection
46 interventional radiology tab selection
47 neuro interventional radiology tab selection
48 modality specific area
49 imaging lab results area
50 mechanical ventilator settings form
51 clinical documentation link
52 Admission orderset
53 free text spaces
54 pre-populatable medical record info field

What is claimed is:

1. A method for enabling the input of clinical orders into a healthcare application, the method comprising the steps of:
(1) Displaying a service line home page with a list of service lines for selection;
(2) Providing a plurality of customizable topic-specific orderset forms for each of the service lines;
(3) Displaying a selected one of the topic-specific orderset forms;
(4) Providing at least one pre-determined, pre-populatable medical record information field on each of the topic-specific orderset forms;
(5) Making multiple, customizable, clinical order selections available for selection; and
(6) Providing contemporary access to a second, ancillary order form or miscellaneous order form.

2. The method according to claim 1, wherein step (6) further comprises displaying a selected one of the ancillary order forms.

3. The method according to claim 1, further comprising the step, after step (6), of: (7) Making at least one free text area available on at least one of the topic-specific orderset forms.

4. The method according to claim 1, further comprising the step, after step (6), of: (8) Displaying a warning if a required information field is not filled in once a computer session utilizing the method is underway.

5. The method according to claim 1, wherein step (6) comprises: Providing access to the ancillary order form and the miscellaneous order form.

6. The method according to claim 1, further comprising the step of:
(9) Providing an option for selecting an additional, miscellaneous order form from a drop-down navigation menu.

7. The method according to claim 1, further comprising the step of:
(10) Providing a Place Orders button for placement of at least one selected clinical order and exiting.

8. The method according to claim 1, wherein the service line home page further comprises at least one one-click frequently used clinical order.

9. The method according to claim 1, wherein the at least one one-click frequently used clinical order is a one-click “Consult Order” button for a particular one of the service lines.

10. A daily rounding method for enabling the input of clinical orders into a healthcare application, the method comprising the steps of:
(1) Providing a “select daily rounding form” option at a point of entry to an order entry collection of a closed loop order navigation system;
(2) Providing an option to select among a plurality of frequently used or miscellaneous order forms, or alternatively proceeding to one of a listing of service line home pages of the system and then displaying a selected one of the service line home pages;
(3) Providing at least one pre-determined, pre-populatable medical record information field on each of the service line home pages; and
(4) Displaying a selected one of the topic-specific orderset forms.

11. The daily rounding method according to claim 10, further comprising the steps of:
(5) Providing at least one pre-determined, pre-populatable medical record information field on each of the topic-specific orderset forms;
(6) Making multiple, customizable, clinical order selections available for selection; and
(7) Providing access to a second, ancillary order form or miscellaneous order form within the closed loop order navigation system.

12. The daily rounding method according to claim 11, further comprising the step, after step (5), of: Displaying a warning if mandatory information fields from a pre-determined list are not filled in.

13. The daily rounding method according to claim 11, further comprising the step of: Providing a Place Orders button by which the clinical order is, or orders are, placed and the closed loop order navigation system is exited.

14. The daily rounding method according to claim 10, wherein the miscellaneous order form options of step (2) comprise the following: (a) Computerized Tomography, Imaging; (b) Vascular Lab, Echocardiogram; and (c) Microbiology.

15. The daily rounding method according to claim 11, wherein, if an additional clinical order form is selected from a drop-down navigation menu on the topic-specific orderset form, a second clinical order form will display after the Place Orders button has been clicked.

16. A system for enabling the input of clinical orders into a healthcare application, the system comprising: (a) a customizable service line home page listing available service lines; (b) a plurality of customizable service specific home pages available on the service line home page; (c) a plurality of customizable topic-specific clinical ordersets related to a selected one of the service lines; (d) a plurality of customizable ancillary order forms available on the service line home page; and (e) a plurality of customizable clinical orders available on each of the topic-specific clinical ordersets or the ancillary order forms; wherein the system enables placement of a plurality of clinical orders in one uninterrupted session.

17. The system according to claim 16, further comprising a plurality of customizable, miscellaneous order forms available on the topic-specific clinical ordersets or the ancillary order forms.

18. The system according to claim 16, wherein the topic-specific clinical ordersets each comprise at least one free text area.

19. The system according to claim 16, wherein the ancillary order forms comprise laboratory, radiology, or electrocardiogram order forms.

20. The system according to claim 16, wherein the service line home page further comprises a one-click “Consult Order” button for a particular one of the service lines.
